(19) United States
(12) Patent Application Publication
(10) Pub. No.: US 2012/0282631 A1
(43) Pub. Date: Nov. 8, 2012

(54) METHOD FOR DIAGNOSIS NON-SMALL-CELL-LUNG-CARCINOMA (NSCLC) AND CLASSIFICATION OF ITS SUBTYPES WITH DIFFERENT COMBINATIONS OF 6 PROTEIN BIOMARKERS

(75) Inventors: Samuel Chun Lap LO, Hong Kong (CN); Yoki Kwok Chu BUTT, Hong Kong (CN); Wallace Woon Fong LEUNG, Hong Kong (CN)

(73) Assignee: THE HONG KONG POLYTECHNIC UNIVERSITY, Hong Kong (CN)

(21) Appl. No.: 13/469,316

(22) Filed: May 11, 2012

Related U.S. Application Data
(63) Continuation-in-part of application No. 12/408,547, filed on Mar. 20, 2009.

(51) Int. Cl. G01N 33/574; C12Q 1/26; G01N 27/26; C12Q 1/48; G01N 27/62; G01N 33/573

(52) U.S. Cl. 435/7.23; 435/7.4; 435/23; 435/7.94; 435/7.1; 435/15; 435/25; 436/86; 436/501

(57) ABSTRACT

A method of identifying and classifying non-small-cell lung carcinoma in a biological sample includes a first test wherein the expression level of at least one biomarker is measured in a sample, the at least one=biomarker is selected from a first group consisting of chaperonin, 2,3-bisphosphoglycerate mutase, thymidine phosphorylase, and minichromosome maintenance deficient protein 5, and comparing the measured expression levels against a normal expression level, wherein a significant difference diagnoses or aids in the diagnosis of non-small cell lung carcinoma; and a second test wherein the expression level of at least one biomarker is measured in the sample, the at least one biomarker is selected from a second group consisting of selenium binding protein and Napsin A, and comparing the measured expression level against a normal expression level, wherein a significant difference confirms or aids in confirmation of Non-Small Cell Carcinoma.

### FIELD OF THE INVENTION

The present invention relates to methods of identifying Non-small-cell Lung Carcinoma in a Biological Sample, and more particularly to identifying and to classification of NSCLC.

### BACKGROUND OF THE INVENTION

Lung carcinoma is the leading cause of cancer deaths worldwide, and non-small cell lung carcinoma (NSCLC) accounts for nearly 85% of all cases of lung cancers. The progression of cancer is usually manifested as uncontrolled growth in cancerous tissues, construction of new blood vessels and invasion into adjacent tissues. These manifestations may bring about concomitant changes in expression of proteins, including their formation, concentrations and interactions with other molecules in the cancerous tissues.

The occurrence of Non-Small Cell Lung Carcinoma is usually diagnosed at a late stage, resulting in high mortality. While early detection of NSCLC is a crucial step to decrease the high mortality rate, there are currently no reliable methods for early detection.

Current diagnostic tests for lung cancer may include chest X-ray, sputum cytology, bronchoscopy, image scans and biopsy. However, these tests can only detect the occurrence of lung cancer at a relatively late stage. Furthermore, because of the limitations imposed on equipment and the establishments required, these methods are not suitable for large-scale diagnostic screening.

### SUMMARY OF THE INVENTION

There is need for a method of identifying Non-Small Cell Lung Carcinoma (NSCLC) in a Biological Sample. Further, it is preferably that the method also provides for classification of NSCLC.

Accordingly, there is disclosed herein a method of identifying and classifying non-small-cell lung carcinoma in a biological sample. The method includes a first test in which the expression level of at least one biomarker is measured in a sample from a human subject. The at least one biomarkers are selected from a first group consisting of chaperonin (CPN), 2,3-bisphosphoglycerate mutase (2,3 BPGM), thymidine phosphorylase (TP), and minichromosome maintenance deficient protein 5 (MCM5). The first test involves comparing the measured expression levels of the at least one biomarker against a normal expression level of the at least one biomarker, wherein a significant difference between the measured expression level of the at least one biomarker selected from the first group compared to the normal expression level diagnoses or aids in the diagnosis of NSCLC.

The method further includes a second test, in which the expression level of at least one biomarker is measured in a sample from a human subject. According to an embodiment, the at least one biomarker is selected from a second group consisting of selenium binding protein (SBP1) and Napsin A (NAPSA). The second test includes comparing the measured expression level of the at least one biomarker against a normal expression level of the at least one biomarker, wherein a significant difference between the measured expression level of the at least one biomarker selected from the second group compared to the normal expression level confirms or aids in confirmation of NSCLC.

According to an embodiment of the invention, the measuring of expression levels in the first and second tests comprises measuring the expression levels of two or more biomarkers from said respective first and second groups. According to an embodiment, the measuring of expression levels for the first and second tests comprises measuring the expression levels of at least two biomarkers in the first group for the first test and at least one biomarker in the second group from the second test. More specifically, the measuring of expression levels for the first test comprises measuring the under-expression of 2,3 BPGM or SBP1, and the measuring of expression levels for the second test comprises measuring the over-expression of CPN, TP, MCM5 or NAPSA. As used herein, a significant difference is an over-expression equal-to or more-than 1.5-fold when the biomarkers are one or more of CPN, TP, MCM5 or NAPSA, and an under-expression equal-to or more-than 1.5-fold, when the biomarkers are one or more of 2,3 BPGM or SBP1.

According to an embodiment of the invention, the first test includes measuring the expression levels of at least one biomarker in a sample from a human subject. Specifically, the first test includes measuring the expression levels of CPN, 2,3 BPGM, TP, MCM5. If the expression levels of CPN, TP and MCM5 are above the normal expression level and the expression level of 2,3 BPGM is below the normal expression level, the sample is diagnosed as having NSCLC.

According to an embodiment of the invention, the second test further aids in classification of the diagnosed NSCLC. Specifically, if the expression level of NAPSA is above the normal expression level and the expression level of SBP1 is the same as or above the normal expression level, the Non-Small Cell Lung Carcinoma is classified as Adenocarcinoma. In the contrary, if the expression level of SBP1 is below the normal expression level and the expression level of NAPSA is the same as or below the normal expression level, the Non-Small Cell Lung Carcinoma is classified as Squamous Cell Carcinoma.

According to an embodiment of the invention, the measuring of expression levels of the at least one biomarker comprises measuring the expression levels of the at least one biomarker using immunoassays. Tissue, interstitial fluid, blood serum, plasma or pulmonary fluid of the subject may be used as a sample for the first and the second tests of the method according the present invention.

### BRIEF DESCRIPTION OF THE DRAWINGS

Preferred embodiments of the invention will now be described with reference to the accompanying drawings in which:

FIGS. 1a-f show differential expressions in immunohistochemical analysis of six biomarkers in non-tumor tissue samples and samples obtained from a patient with Adenocarcinoma (ADC) and Squamous Cell Carcinoma (SCC),

FIG. 2 depicts the enlarged 2-dimensional gel regions showing chaperonin expressed in non-tumor and tumor tissues of three patients.

FIG. 3 depicts the enlarged 2-dimensional gel regions showing 2,3-bisphosphoglycerate mutase expressed in non-tumor and tumor tissues of three patients.

FIG. 4 depicts the enlarged 2-dimensional gel regions showing thymidine phosphorylase expressed in non-tumor and tumor tissues of three patients.

FIG. 5 depicts the enlarged 2-dimensional gel regions showing selenium binding protein 1 expressed in non-tumor and tumor tissues of three patients.

FIG. 6 depicts the enlarged 2-dimensional gel regions showing minichromosome maintenance deficient protein 5 variant expressed in non-tumor and tumor tissues of three patients.

FIG. 7 depicts the enlarged 2-dimensional gel regions showing napsin A aspartic peptidase expressed in non-tumor and tumor tissues of three patients.

FIG. 8a is a figure illustrating results of the western blot analysis of chaperonin in tumor tissues and the adjacent non-tumor tissues from 10 NSCLC patients.

FIG. 8b depicts the fold change in the expression level of chaperonin in lung tumor tissues, compared with the adjacent non-tumor lung tissues.

FIG. 9 illustrates a method of identifying Non-Small Cell Lung carcinoma in a biological sample,

FIG. 10 illustrates a method of classifying Adenocarcinoma from Squamous Cell Carcinoma in a biological sample from a patient with Non-Small-Cell Lung carcinoma, and

FIG. 11 illustrates a method of identifying and classifying non-small-cell lung carcinoma in biological samples under investigation.

### DESCRIPTION OF THE PREFERRED EMBODIMENTS

Immunohistochemistry refers to a process of localizing protein antigens in cells of a paraffin formalin-fixed tissues section by their specific binding with the corresponding antibodies. One aspect of the current invention uses immunohistochemistry as a tool to carry out tests with a panel of six biomarkers and using the results of the immunohistochemistry on the six biomarkers as an indicator for the occurrence of non-small cell lung carcinoma (NSCLC) and classification of the subtypes of NSCLC in biological samples.

With the use of a proteomic approach and subsequent verification by immunohistochemistry, the six proteins were shown to have differential expression in lung cancerous tissues. The term “differential expression” refers to the difference in protein expression levels between normal lung tissues and cancerous lung tissues. The change in expression levels of these six proteins can be used as biological indicators for the occurrence of Non-Small Cell Lung Carcinoma. For early detection and large scale screening purposes, the sensitivity and specificity of a single biomarker remains a challenge, therefore, the use of a combination or a panel of biomarkers is superior as it offers improved accuracy. Hence, a combination of these six “cancer” biomarkers is useful for early identification of Non-Small Cell Lung Carcinoma and subsequent classification of the subtypes of Non-Small Cell Lung Carcinoma.

In a preferred embodiment of the invention, Non-Small Cell Lung Carcinoma can be identified in a biological sample obtained from a patient by comparing the expression levels of two or more biomarkers in the sample, where the biomarkers are selected from a group consisting of chaperonin (CPN), 2,3-bisphospgolycerate mutase (2,3 BPGM), thymidine phosphorylase (TP), selenium binding protein 1 (SBP1), minichromosome maintenance deficient protein 5 (MCM5) and napsin A (NAPSA). The method involves obtaining a fresh or previously obtained biological sample from a patient and measuring the expression levels of two or more of the above-mentioned biomarkers. The measured expression levels of the two or more biomarkers are then compared against normal expression levels of these proteins and a significant difference between the measured expression levels of the two or more biomarkers compared to the normal expression levels is indicative of the occurrence of non-small cell lung carcinoma.

“Normal expression level” is defined by a measurement or a ratio in a normal biological sample, and is a standardized value obtained from a group of healthy subjects. The ratio may be calculated as:

Ratio=(optical density of the protein spot*spot area*spot volume in tumor tissue)/(optical density of the same protein spot*spot area*spot volume in non-tumor tissue)

As used herein, a normal biological sample is defined as a sample from a normal individual without any known symptoms and observable abnormalities. A healthy subject, or a normal individual, is defined as a person without any known symptoms and observable abnormalities, such as NSCLC. In clinical practice, the norm is usually defined as the average of a large cohort of normal subjects (e.g. 50 people or as big as practical).

The biological sample can be tissue, interstitial fluid, blood, serum, plasma or pulmonary fluid, and the expression levels of two or more biomarkers can be measured using immunoassays or other known methods. Examples of immunoassays that may be used to determine expression levels of the biomarkers include, but are not limited to, sandwich immunoassays (ELISA or fluorescence-based immunoassays), western blotting, immunohistochemistry, nephelometry, SPR biosensors, SELDI-based immunoassays, and various kinds of mass spectrometry based methodologies.

A significant difference is preferably, although not essentially, clarified as a difference equal-to or more-than 1.5-fold. For CPN, TP, MCM5 or NAPSA biomarkers, the significant difference is an over-expression, preferably equal-to or more-than 1.5-fold. For 2,3 BPGM or SBP1 biomarkers, the difference is an under-expression, preferably equal-to or more-than 1.5-fold.

The biomarkers CPN, 2,3 BPGM, TP, MCM5 are used to identify non-small cell lung carcinoma in the biological samples obtained from patients and the biomarkers SBP1 and NAPSA are used to classify or aid in classifying the subtypes of non-small cell lung carcinoma. If the expression levels of CPN, TP and MCM5 are above the normal expression levels, and the expression level of 2,3 BPGM is below the normal expression level, then non-small cell lung carcinoma is present in the patient providing the biological sample.

Subsequent to the above, if the expression level of NAPSA is above the normal expression level and the expression level of SBP1 is the same as or above the normal expression level, then the Non-Small Cell Lung Carcinoma can be classified as Adenocarcinoma. In the contrary, if the expression level of SBP1 is below the normal expression level and the expression level of NAPSA is the same as or below the normal expression level, then the Non-Small Cell Lung Carcinoma can be classified as Squamous Cell Carcinoma. Alternatively, Non-Small Cell Lung Carcinoma can be identified in patients using conventional methods before this method is employed to classify the subtypes of Non-Small Cell Lung Carcinoma as either Adenocarcinoma or Squamous Cell Carcinoma.

Accordingly, in an alternative embodiment of the invention, the type of non-small-cell lung carcinoma can be identified in a biological sample. Firstly, non-small cell lung carcinoma is identified in patients using conventional methods, or in a biological sample provided by such patients using the CPN, 2,3 BPGM, TP, MCM5 biomarkers as mentioned previously. Subsequently, the expression levels of selenium binding protein 1 (SBP1) and napsin A (NAPSA) in the sample are measured. The measured expression levels of the biomarkers are compared against their normal expression levels, and a significant difference between the measured expression levels and the normal expression level classifies or aids in classifying the subtype of non-small cell lung carcinoma. If the expression level of NAPSA is above the normal expression level and the expression level of SBP1 is the same as or above the normal expression level, then the non-small cell lung carcinoma can be classified as Adenocarcinoma. If the expression level of SBP1 is below the normal expression level and the expression level of NAPSA is the same as or below the normal expression level, then the non-small cell lung carcinoma can be classified as Squamous Cell Carcinoma. The biological sample can be tissue, interstitial fluid, blood, serum, plasma or pulmonary fluid, and the expression levels of two or more biomarkers can be measured using immunoassays or other known methods. A significant difference is preferably, although not essentially, clarified as a difference equal-to or more-than 1.5-fold.

According to an alternative embodiment of the invention, there is provided a method for detecting the level/presence of autoimmune antibodies against a group of biomarkers comprising TP, CPN, SBP1, MCM5, NAPSA, as an indicator for the occurrence of Non-Small Cell Lung Carcinoma in serum samples. Antibodies are produced by the body as a result of immune response against foreign antigens, such as proteins or carbohydrates presented on the surface of invading organisms. Often, however, the immune system recognizes normal body constituents as antigens and generates antibodies against these “self-antigens”. This process is known as autoimmunity, and the antibodies generated against “self-antigens” are termed autoimmune antibodies. Novel proteins or over-expression of normal proteins, such as the above-mentioned biomarkers, are often present in cancerous cells, this triggers an autoimmune response within the body which results in the presence of autoimmune antibodies against these carcinoma antigens or biomarkers. The presence of these autoimmune antibodies is therefore a useful indicator for the occurrence of carcinomas.

In another embodiment of the invention, non-small cell lung carcinoma can be identified in a serum sample obtained from a patient by detecting the presence of autoimmune antibodies against biomarkers chosen from a group comprising TP, CPN, SBP1, and Enolase-1. The method involved obtaining serum samples from a patient and detecting the presence of autoimmune antibodies against the above-mentioned biomarkers. The presence of one or more autoimmune antibodies against the biomarkers chosen from the above-mentioned group indicates the occurrence of non-small cell lung carcinoma in patients providing the serum samples. An example of techniques that can be used to detect the presence of autoimmune antibodies is 1D- and 2D-western blot.

### 1. Biomarkers

The biomarkers used in this invention are selected from a group consisting of chaperonin (CPN), 2,3-bisphospgolycerate mutase (2,3 BPGM), thymidine phosphorylase (TP), selenium binding protein 1 (SBP1), minichromosome maintenance deficient protein 5 (MCM5) and napsin A (NAPSA).

### 1.1 Chaperonin

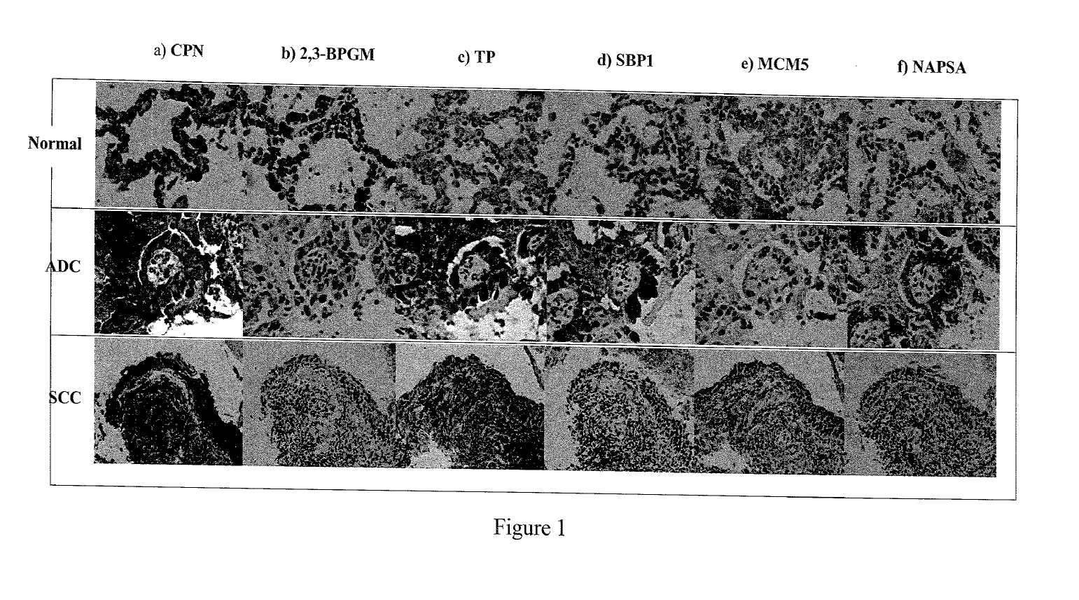

Chaperonin (CPN) is up-regulated in lung tumor tissues. Chaperonin may also be known as chaperonin 60, heat shock protein 60, mitochondrial matrix protein P1, and P60 lymphocyte protein. Chaperonin has an accession number of P10809, and a molecular weight of 61.0 kDa. FIG. 1a showed enlarged pictures of immunohistochemical staining results of chaperonin in tissues samples from a non-cancer normal subject or patient with either Adenocarcinoma (ADC), or Squamous Cell Carcinoma (SCC). Chaperonin, or heat shock protein 60, is a highly conserved protein mostly localized in the mitochondrial matrix. Chaperonin may play a role in the regulation of a variety of cellular functions. The primary function is to assist in mitochondrial protein folding, unfolding and degradation. Besides, chaperonin may also be associated with the regulation of protein turnover and the cellular redox state.

### 1.2 2,3-Bisphosphoglycerate Mutase

2,3-bisphosphoglycerate mutase (2,3 BPGM) is down-regulated in lung tumor tissues. 2,3 BPGM may also be known as 2,3-bisphosphoglycerate synthase and 2,3-bisphosphoglycerate synthase phosphatase. 2,3 BPGM has an accession number of P07738, and a molecular weight of 30.0 kDa. Figure lb showed enlarged pictures of immunohistochemical staining results of 2,3-BPGM in tissue samples from a non-cancer normal subject or patients with either Adenocarcinoma (ADC) or Squamous Cell Carcinoma (SCC). 2,3-bisphosphoglycerate mutase (BPGM) catalyzes the conversion of 1,3-bisphosphoglycerate (1,3-BPG) to 2,3-bisphosphoglycerate (2,3-BPG). In mammalian tissues, BPGM may have 2-phosphoglycolate-stimulated 2,3-bisphosphoglycerate phosphatase (BPGP) activity. The final product of BPGM, 2,3-BPG, may play an important role in erythrocytes, in which it acts as an allosteric inhibitor of hemoglobin. The binding of 2,3-BPG decreases hemoglobin's affinity for oxygen and facilitates the transfer of oxygen to tissues. Excess amounts of 2,3-BPG re-enter the glycolytic pathway through a phosphatase-catalyzed conversion to glycerate-3-phosphate.

1.3 Thymidine Phosphorylase

Thymidine phosphorylase (TP) is up-regulated in lung tumor tissues. Thymidine phosphorylase may also be known as gliostatin, blood platelet-derived endothelial cell growth factor. Thymidine phosphorylase has an accession number of P19971, and a molecular weight of 49.9 kDa. FIG. 1c showed enlarged pictures of immunohistochemical staining results of TP in tissue samples from a non-cancer normal subject or patients with either Adenocarcinoma (ADC) or Squamous Cell Carcinoma (SCC). In the presence of inorganic orthophosphate, thymidine phosphorylase may catalyze the reversible phosphorolysis of thymidine and 2-deoxyuridine to their corresponding bases, thymine and 2-deoxyribose-1-phosphate. These produced molecules may then be utilized as carbon and energy sources in the rescue of pyrimidine bases for nucleotide synthesis. Besides, thymidine phosphorylase may have both angiogenic and chemotactic properties.

### 1.4 Selenium-Binding Protein 1

Selenium-binding protein 1 (SBP1), is down-regulated in lung tumor tissues. Selenium binding protein 1 may also be known as 56 kDa selenium-binding protein. SBP1 has an accession number of Q13228, and a molecular weight of 52.4 kDa. FIG. 1d showed enlarged pictures of immunohistochemical staining results of SBP1 in tissue samples from a non-cancer normal subject or patients with either Adenocarcinoma (ADC) or Squamous Cell Carcinoma (SCC) Selenium is an essential micronutrient that exhibits potent anticarcinogenic properties, and deficiency of selenium may cause carcinomas and certain neurologic diseases.

### 1.5 Minichromosome Maintenance Deficient Protein 5

Minichromosome maintenance deficient protein 5 (MCM5) is up-regulated in lung tumor tissues. MCM5 variant may also be known as minichromosome maintenance complex component 5 DNA replication licensing factor, MCM5 minichromosome maintenance deficient 5. MCM 5 has an accession number of Q53FG5, and a molecular weight of 82.2 kDa. Figure le showed enlarged pictures of immunohistochemical staining results of MCM5 in tissue samples from a non-cancer normal subject or patients with either Adenocarcinoma (ADC) or Squamous Cell Carcinoma (SCC). Minichromosome maintenance (MCM) protein family consists of six members (MCM2-7) and is a conserved group of DNA-binding proteins. MCM proteins may be specifically up-regulated in the transition from the G0 to G1/S phases of the cell cycle, during which they may be involved in the regulation of DNA synthesis

### 1.6 Napsin A

Napsin A (NAPSA) is up-regulated in lung tumor tissues. Napsin A may also be known as Napsin A aspartic peptidase and napsin-1, aspartyl protease 4. Napsin A has an accession number of Q8WWD9, and a molecular weight of 45.4 kDa. Figure if showed enlarged pictures of immunohistochemical staining results of NAPSA in tissue samples from a non-cancer normal subject or patients with either Adenocarcinoma (ADC) or Squamous Cell Carcinoma (SCC). Napsin A aspartic peptidase is a member of the aspartic protease family, which is expressed predominantly in the cytoplasm of cells of lung (type II pneumocytes) and kidney.

### 2. Panel of Biomarkers

According to a first aspect of the invention, there is provided a method for early identification of non-small cell lung carcinoma using a panel of at least two biomarkers chosen from a group comprising CPN, TP, MCM5 and 2,3 BPGM. A method of identifying the occurrence of non-small cell lung cancer may involve measuring the expression levels of two or more of the above-mentioned biomarkers and comparing the measured expression levels against the normal expression levels of these proteins. When the expression level of one or more of CPN, TP, and MCM5 is 1.5 fold above the normal expression level, and the expression level of 2,3BPGM is 1.5 fold below the normal expression level, the biological sample may be interpreted as being cancerous.

Subsequent to the identification of Non-Small Cell Lung Carcinoma in biological sample, there is provided a method for classifying the subtypes of non-small cell lung cancer using a panel of two biomarkers consisting of NAPSA and SBP1. This method involves measuring the expression levels of NAPSA and SBP1 and comparing the measured expression levels against the normal expression levels of these biomarkers. If the expression level of NAPSA is above the normal expression level and the expression level of SBP1 is the same as or above the normal expression level, the non-small cell lung carcinoma can be classified as Adenocarcinoma. If the expression level of SBP1 is below the normal expression level and the expression level of NAPSA is the same as or below the normal expression level, then the non-small cell lung carcinoma can be classified as Squamous Cell Carcinoma.

### 3. Method of Use of Biomarkers

The protein biomarkers may be detected quantitatively and semi-quantitatively by different antibody-based techniques, different types of mass spectrometry (SELDI MS, MALDI TOF MS and tandem MS), or other means in various biological samples (including but not limited to biopsy tissues, tumor tissues and blood fluids). If the biological samples already contain the target biomarkers in soluble form, (e.g. serum, plasma, tissue fluids, secretion, biopsy fluid etc.), no additional protein extraction method is needed. Subsequently, antibody-based detection techniques may included different types of immunoassays, conventional or tissue microarray immunohistochemical analysis. Examples of immunoassays include sandwich immunoassays (ELISA or fluorescence-based immunoassays), nephelometry, and biosensors SELDI-based immunoassays.

3.1 Protein Extraction from Tissue Samples

To prepare protein extracts from tissue samples, the entire tissue is homogenized in phosphate buffer containing a protease inhibitors cocktail (available from Sigma, U.S.A.). After homogenization, supernatant containing proteins is isolated by centrifugation at 16000 g for 15 minutes at 4° C. and can be stored at -80° C. until use. Protein concentration may be estimated using the Bradford protein assay (available from Bio-Rad Laboratories Ltd. in Hercules, Calif., U.S.A.).

### 3.2 Detection by Antibody-Based Techniques

The presence of protein biomarkers and their expression levels in the biological samples can be detected with immunohistochemical methods, or measured quantitatively by different types of immunoassays. Both of which are antibody-based techniques that require a capture reagent, such as bio-specific antibodies, to interact specifically with the entire biomarker or a polypeptide of the biomarker (referred to as antigens).

Antibodies may be monoclonal or polyclonal, and may refer generally to any immunologic capture agent such as IgG, IgM, IgA, IgD and IgE. Monoclonal antibodies may be used to bind to one site of a particular molecule, and therefore may provide a more specific and accurate result. Antibodies can be produced by immunizing animals or poultry with the entire antigen (protein biomarker) or a specific polypeptide of the antigen (either naturally occurring or synthetic). Examples of immunizing animals include rats, mice, and rabbits. Examples of poultry include hens. The antigen used for immunization may also be isolated from the samples or synthesized by recombinant protein technology.

Primary antibodies provide the specific recognition for detecting or quantifying the protein biomarker, while the secondary detection system utilizes enzymes (e.g. enzyme immunoassay (EIA)), radioisotopes (e.g. 1-125 radioimmunoassay (RIA)), magnetic labels (e.g. magnetic immunoassay (MIA)), fluorescence, or other conjugated secondary antibodies.

With the use of specific antibodies, an immunohistological stained tissue section for pathological examination can be obtained by conventional and/or tissue microarray immunohistochemical analysis. In addition, tissues can also be extracted for the liberation of protein biomarkers for western blot or dot-blot assays. In this technique, which is based on the use of cationic solid phases, quantitation of protein biomarkers can be accomplished by comparing isolated biomarker standards to a known concentration. A molar concentration of a protein biomarker may aid to set the standard values of the biomarker content for different tissues, fecal matter or body fluids (e.g. serum, plasma, urine, synovial fluid, spinal fluid). Finally, the normal appearance of biomarker amounts may be set using values from healthy subjects, and can be compared against those obtained from a test subject. Other immuno-techniques include immunoelectrophoresis, agglutination, nephelometry, turbidimetry and biosensors, and SELDI-based mass spectrometry.

### 3.3 Detection by Mass Spectrometry

In addition to antibody-based techniques, the protein biomarkers can also be detected and quantified by mass spectrometry. Mass spectrometry is a method that employs a mass spectrometer to detect ionized protein markers or ionized peptides as digested from the protein markers (called peptide-mass-fingerprints) by measuring the mass-to-charge ratio (m/z). For example, analytes are introduced into a sample inlet and ionized in an ionization source, and the ionized analytes are introduced into mass analyzers for mass separation before their mass being measured by a detector.

Differences in the sample inlet, ion source, and mass analyzer may generally define the type of instrument and its capacities. For instance, a sample inlet may include a capillary-column liquid chromatography source, or may include a direct probe or a stage as used in matrix-assisted laser desorption. Examples of the ion source may include an electrospray, a nanospray, a microspray, and a matrix-assisted laser desorption. Example of the mass analyzers may include a quadrupole mass filter, an ion trap mass analyzer, and a time-of-flight mass analyzer. Preferably, matrix-assisted laser deionization/ionization time-of-flight (MALDI-TOF) mass spectrometry, tandem mass spectrometry, and surface enhanced laser desorption/ionization (SELDI) mass spectrometry may be used.

A mass spectrometric method for identifying a lung cancer biomarker or the corresponding peptide includes providing a biological sample from a subject having lung cancer, concentrating digested peptides belonging to protein biomarkers in the biological sample, separating one or more of the digested peptides, and subjecting the separated peptides to mass spectrometry to obtain primary amino acid sequences of at least one of the separated peptides. The amount of at least two biomarkers in the biological sample from a subject may be measured using mass spectrometry. One or both of an increase in the amount of signal at the m/z ratio values specific to the digested peptides from an up-regulated protein biomarker, and a decrease in the amount of signal at the m/z ratio values specific to the digested peptides from a down-regulated protein biomarker in the biological sample as compared to a non-lung cancer reference amount of the signals may indicate that the subject may have lung cancer. SELDI is a technique based on the pretreatment of a biological fluid or tissue extract with various protein/peptide chips, performing protein extractions based on hydrophobic, ion-exchange, metal binding, or other interactions. The bound proteins/peptides may then be subjected to mass spectrometric analysis.

### 3.4 Detection of Protein Expression Levels by 2D Gel Electrophoresis and Image Analysis

### A. Protein Separation by 2D Gel Electrophoresis

2D gel electrophoresis is performed on PROTEIN IEF cell and PROTEAN II XL system (available from Bio-Rad Laboratories Ltd in Hercules, Calif., U.S.A.). Equal protein loading corresponding to tissues samples can be normalized by relative expression of housekeeping protein—β-lactin prior to 2D gel electrophoresis. Proteins contained within the supernatant may be separated in a first dimension by Isoelectric Focusing (IEF) followed by separation in a second dimension by sodium dodecyl sulfate polyacrylamide gel electrophoresis (SDS-PAGE). For IEF, protein samples added to the rehydration buffer (2M thiourea, 7M urea, 4% (v/v) CHAPS, 10% (v/v) glycerol, 64 mM DTT and 1% IPG buffer) can be rehydrated actively onto pre-cast 18 cm linear pH gradient 4-7 IPG strips (available from Bio-Rad Laboratories Ltd.), after which the subsequent IEF is performed at 20° C. with a PROTEAN IEF cell (available from Bio-Rad Laboratories Ltd.) with the following Voltages (V): 1 hour at 500V, 1 hour at 1000V, 2 hours at 4000V, 4 hours at 8000V, resulting in a total voltage of 100 kVhr.

Following IEF, the IPG strips is equilibrated with 1% (w/v) DTT in the equilibrium buffer (50 mM Tris-HCl (pH8.8), 6 M urea, 39% (v/v) glycerol, 2% (w/v) SDS and 0.006% (w/v) bromophenol blue) for 15 minutes and subsequently in a solution of 1% (w/v) iodoacetamide in the same equilibrium buffer for 15 minutes. The IPG strips may be rinsed with ddH₂O and placed on top of a 10% SDS gel and sealed with 0.5% agarose in electrophoresis buffer for SDS-PAGE. Electrophoresis may be performed for the first 30 minutes at 15 mA/gel followed by 30 mA/gel until the bromophenol blue dye front reaches the lower end of the gel using a PROTEN II xi 2D cell electrophoresis unit (available from Bio-Rad Laboratories Ltd.). Proteins may be visualized by mass spectrometry or compatible silver staining image analysis.

### B. Visualization of Protein Spots and Image Analysis

After SDS-PAGE, the gel is fixed for 1 hour initially in fixation solution containing 40% (v/v) ethanol and 10% (v/v) acetic acid, following which it is sensitized in a solution containing 30% (v/v) ethanol, 0.2% (w/v) sodium thiosulphate, 6.8% (w/v) sodium acetate and 0.125% (v/v) glutaraldehyde for 30 minutes, and then washed with distilled water (for example, 3 times for 5 minutes each).

Subsequently the gel is stained for 20 minutes in 0.25% (w/v) silver nitrate with 0.015% (v/v) formaldehyde before washing with distilled water again (for example 2 times for 1 minutes each). The gels can then be developed in 2.5% (w/v) sodium carbonate containing 0.0074% (v/v) formaldehyde to visualize the protein spots. The development is typically stopped using 1.5% (w/v) EDTA.

Sliver-stained gels are scanned at 300 dots per inch resolution using a conventional scanner (e.g. available from Epson Perfection 1200U). All spot detection and spot comparisons are performed using Melanie 3.08 (available from Gene-Bio in Switzerland). Spot intensity may be normalized by plotting the optical density of each paired and matched protein spots in two comparing gels in a scatter plot. The differential expression can be confirmed when spots for a particular biomarker are consistently present on both gels. Moreover, the significance may be regarded only when the difference in spot optical density is at least by 1.5 fold.

### 4. Detection of the Presence of Autoimmune Antibodies Against a Panel of Biomarkers in Blood Serum Using 2D Western Blotting

### 4.1 Western Blotting

Protein samples extracted from the lung tissues of patients known to suffer from Non-Small Cell Lung Carcinoma, or protein samples containing a mixture of biomarkers for Non-Small Cell Lung Carcinoma are be resolved on two-dimensional SDS-PAGE and electro-blotted onto a nitro-cellulose (NC) membrane. After electro-blotting, the NC membrane is blocked with 5% (w/v) skim milk in Tris buffer saline with 0.05% (v/v) Tween 20 (TBST) for 2 hours at room temperature. Subsequently, the membrane is incubated with serum samples obtained from a test subject for 2 hours at room temperature. After six washings, the membrane is then be incubated with horse-radish peroxidase-conjugated antibodies for 1 hour at room temperature. Finally, the signal is visualized with Super Signal ECL Kit (available from Promega in the U.S.A.). The intensities of the corresponding immunoreactive spots can be measured as the black light units (BLU). If immunoreactive spots appear at the corresponding positions of the biomarkers on the gel, autoimmune antibodies are present in the sera of the test subject, which is indicative of the occurrence of Non-Small Cell Lung Carcinoma.

The method described above may be used to detect the presence of early stages of NSCLC for screening, diagnostic and prognostic purposes. The method may be further developed for use in the determination of the efficacy of treatment on lung cancer due to surgery, radiation and chemotherapy, as well as further the design of targeted drug for cancer treatment.

### EXAMPLES

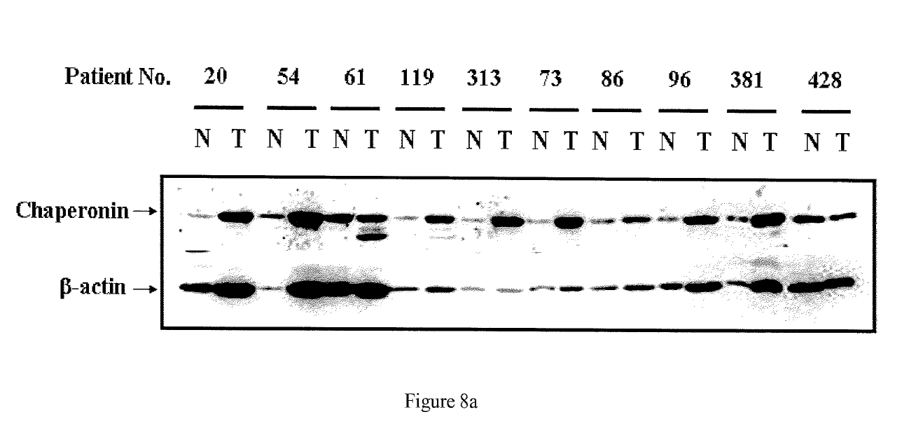

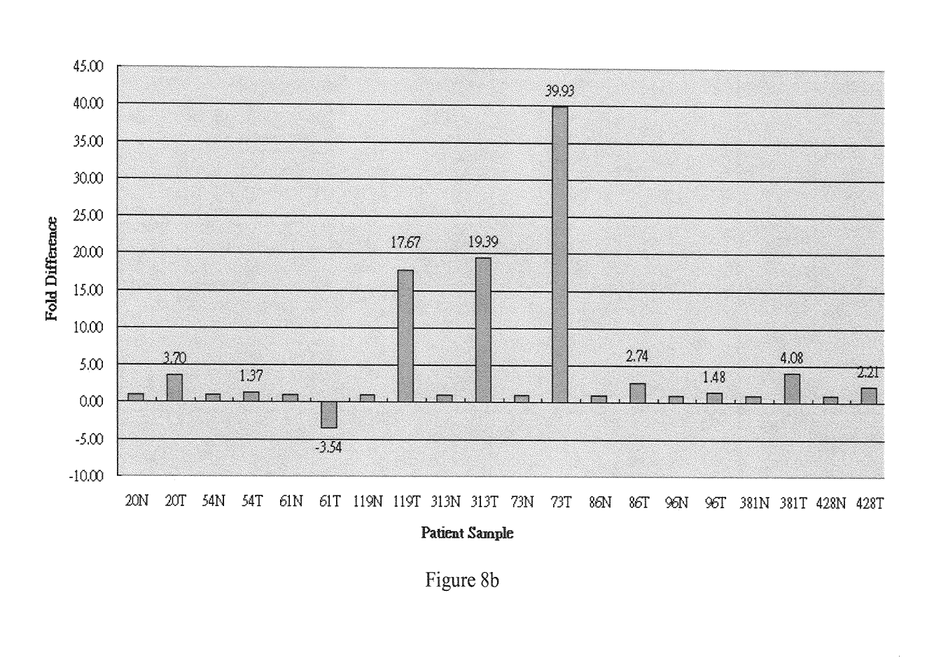

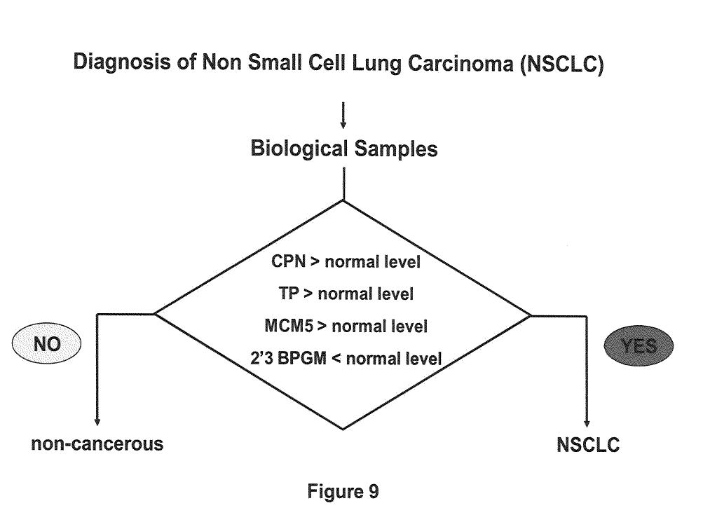

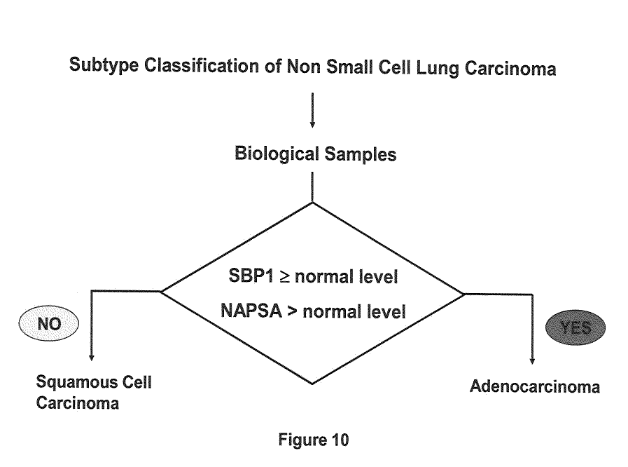

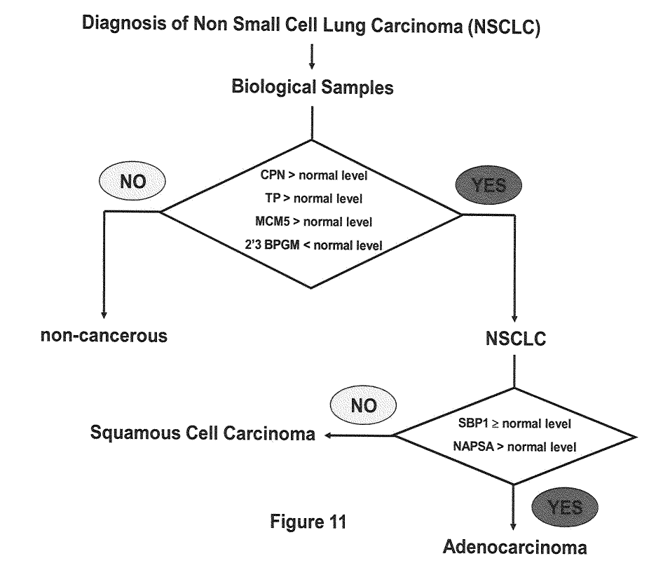

Four examples of the invention are given in FIGS. 1-11 and Table 1.

### Example 1

Table 1 is a summary of the expression of CPN, 2,3BPGM, TP, SBP1, MCM5 and NAPSA in 19 Non-Small Cell Lung Carcinoma specimens including ADC and SCC.

| | Immunohistochemical Markers | | | | | | | | | | | |
|---|---|---|---|---|---|---|---|---|---|---|---|---|
| | CPN | | TP | | MCM5 | | NAPSA | | 2′3-BPGM | | SBP1 | |
| | Inten- | % of | Inten- | % of | Inten- | % of | Inten- | % of | Inten- | % of | Inten- | % of |
| Samples | sity | cells | sity | cells | sity | cells | sity | cells | sity | cells | sity | cells |
| Control | + | >75 | − | 0 | − | 0 | − | 0 | +++ | >75 | ++ | >75 |
| Lung | | | | | | | | | | | | |
| adenocarcinomas | | | | | | | | | | | | |
| ADC4 | +++ | >75 | +++ | 51-75 | + | 1-25 | ++ | >75 | − | 0 | − | 0 |
| ADC5 | +++ | >75 | +++ | >75 | ++ | 51-75 | ++ | >75 | + | 26-50 | +++ | 51-75 |
| ADC6 | +++ | >75 | ++ | 51-75 | − | 0 | ++ | >75 | + | >75 | − | 0 |
| ADC7 | +++ | >75 | +++ | >75 | − | 0 | +++ | >75 | − | 0 | − | 0 |
| ADC8 | +++ | >75 | +++ | 26-50 | + | 1-25 | +++ | .75 | + | 26-50 | ++ | 51-75 |
| ADC1 | +++ | >75 | ++ | >75 | ++ | 1-25 | − | 0 | − | 0 | − | 0 |
| ADC10 | +++ | >75 | +++ | >75 | + | 1-25 | +++ | >75 | + | 1-25 | ++ | 51-75 |
| ADC11 | +++ | >75 | +++ | 26-50 | ++ | 51-75 | ++ | >75 | + | 1-25 | +++ | >75 |
| ADC12 | +++ | >75 | +++ | >75 | + | 1-25 | +++ | >75 | + | >75 | ++ | >75 |
| ADC13 | +++ | >75 | +++ | 51-75 | ++ | 26-50 | ++ | 51-75 | +++ | >75 | + | 1-25 |
| Squamous cell | | | | | | | | | | | | |
| carcinomas | | | | | | | | | | | | |
| SCC1 | +++ | >75 | ++ | 1-25 | + | 51-75 | − | 0 | − | 0 | − | 0 |
| SCC2 | +++ | >75 | +++ | 51-75 | +++ | >75 | − | 0 | − | 0 | − | 0 |
| SCC3 | +++ | >75 | ++ | 26-50 | ++ | 51-75 | − | 0 | − | 0 | − | 0 |
| SCC4 | +++ | >75 | +++ | 51-75 | + | 1-25 | − | 0 | − | 0 | − | 0 |
| SCC5 | +++ | >75 | ++ | >75 | + | 1-25 | − | 0 | + | 1-25 | − | 0 |
| SCC6 | +++ | >75 | +++ | 1-25 | + | 51-75 | − | 0 | − | 0 | − | 0 |
| SCC7 | +++ | >75 | ++ | 51-75 | + | 1-25 | − | 0 | + | 1-25 | − | 0 |
| SCC8 | +++ | >75 | +++ | >75 | ++ | 51-75 | ++ | 1-25 | + | 1-25 | + | 26-50 |
| SCC9 | +++ | >75 | +++ | >75 | +++ | >75 | + | 1-25 | − | 0 | + | 1-25 |

### Example 2

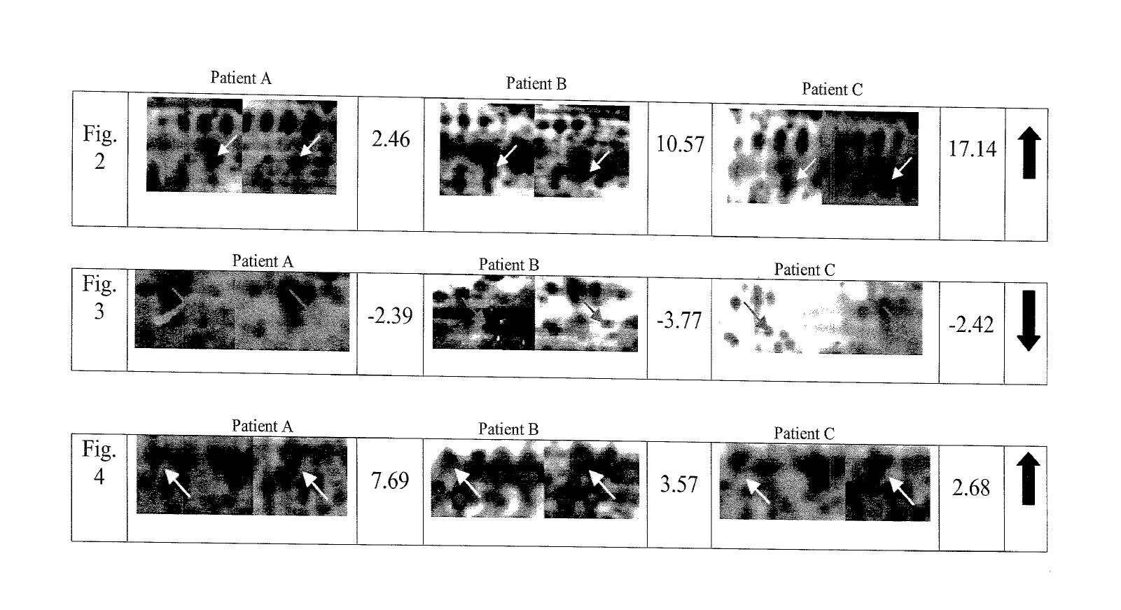

Referring to FIG. 2, a combination or a panel of 4 biomarkers, including CPN, 2,3 BPGM, TP and MCM5, is used to indicate whether the biological sample indicative of NSCLC. For example, if the expression of CPN, TP and MCM5 is larger than the normal level and the expression of 2,3 BPGM is lower than the normal level, the sample is considered to be tested positive for NSCLC. Otherwise, the biological sample is not indicative of NSCLC. The normal level is defined by the expression level of the protein markers in normal lung tissues.

### Example 3

Referring to FIG. 3, another combination or panel of two protein biomarkers is used to classify the subtypes of NSCLC as either lung adenocarcinoma (ADC) or squamous cell carcinoma (SCC). The two protein biomarkers are SBP1 and MCM5. The NSCLC is classified as ADC if the expression levels of SBPI and NAPSA are above or equal to the normal level, whereas it is classified as SCC if the expression levels of SBPI and NAPSA are below the normal level.

### Example 4

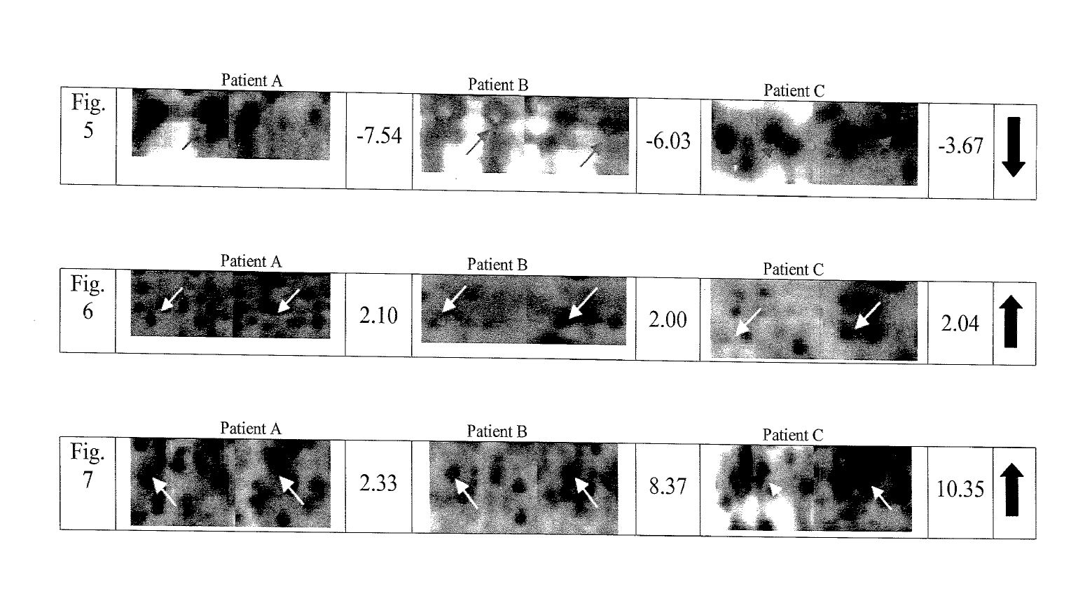

Referring to FIG. 4, identification and classification is carried out in 2 stages, with the first outcome implemented followed by the second outcome in sequence. For example, a combination or a panel of 4 biomarkers, including CPN, 2,3 BPGM, TP and MCM5, is used to infer whether the biological sample is indicative of NSCLC and then the subtype of NSCLC is classified by the second outcome. The combination or panel of two protein biomarkers, SBP1 and MCM5, is used to classify the subtypes of NSCLC as either lung adenocarcinoma (ADC) or squamous cell carcinoma (SCC).

Although exemplary embodiments of the invention have been described, it should be understood that the biomarkers and method are not limited to such embodiments. The skilled addressee will understand that modifications may be made to the current invention and elements described herein replaced by known equivalents.

1. A method of identifying and classifying non-small-cell lung carcinoma in a biological sample, the method comprising:
a first test wherein the expression level of at least one biomarker is measured in a sample from a human subject, wherein the at least one biomarker is selected from a first group consisting of chaperonin (CPN), 2,3-bisphosphoglycerate mutase (2,3 BPGM), thymidine phosphorylase (TP), and minichromosome maintenance deficient protein 5 (MCM5);
comparing the measured expression levels of the at least one biomarker against a normal expression level of the at least one biomarker, wherein a significant difference between the measured expression level of the at least one biomarker selected from the first group compared to the normal expression level diagnoses or aids in the diagnosis of non-small cell lung carcinoma;
a second test wherein the expression level of at least one biomarker is measured in a sample from a human subject, wherein the at least one biomarker is selected from a second group consisting of selenium binding protein (SBP1) and Napsin A (NAPSA); and
comparing the measured expression level of the at least one biomarker against a normal expression level of the at least one biomarker, wherein a significant difference between the measured expression level of the at least one biomarker selected from the second group, compared to the normal expression level, confirms or aids in confirmation of non-small cell lung carcinoma.

2. The method of claim 1 wherein said second test further aids in classification of the diagnosed non-small cell lung carcinoma.

3. The method of claim 1 wherein the measuring of expression levels in the first and second tests comprises measuring the expression levels of two or more biomarkers from the respective first and second groups.

4. The method of claim 1 wherein the measuring of expression levels for the first and second tests comprises measuring the expression levels of at least two biomarkers in the first group for the first test and at least one biomarker in the second group from the second test.

5. The method of claim 1, wherein the measuring of expression levels for the first test comprises measuring the under-expression of 2,3 BPGM or SBP1.

6. The method of claim 1, wherein the measuring of expression levels for the second test comprises measuring the over-expression of CPN, TP, MCM5, and NAPSA.

7. The method of claim 1, wherein the measuring of expression levels of the at least one biomarker comprises measuring the expression levels of the at least one biomarker using immunoassays.

8. The method of claim 1, wherein the sample comprises a sample selected from a group consisting of tissue, interstitial fluid, blood serum, plasma, and pulmonary fluid of the subject.

9. The method of claim 1, wherein the significant difference is an over-expression equal-to or more-than 1.5-fold when the biomarkers are one or more of CPN, TP, MCM5, and NAPSA.

10. The method of claim 1, wherein the significant difference is an under-expression equal-to or more-than 1.5-fold, when the biomarkers are one or more of 2,3 BPGM and SBP1.

11. The method of claim 1, wherein the first test conducted to measure the expression levels of at least one biomarker in a sample from a human subject comprises measuring the expression levels of CPN, 2,3 BPGM, TP, MCM5 and diagnosing the sample as having non-small cell lung carcinoma when the expression levels of CPN, TP and MCM5 are above the normal expression level and the expression level of 2,3 BPGM is below the normal expression level.

12. The method of claim 1 comprising classifying non-small cell lung carcinoma as Adenoarcinoma when the expression level of NAPSA is above the normal expression level and the expression level of SBP1 is the same as or above the normal expression level.

13. The method of claim 1 further comprising classifying non-small cell lung carcinoma as Squamous Cell Carcinoma when the expression level of SBP1 is below the normal expression level and the expression level of NAPSA is the same as or below the normal expression level.

14. The method of claim 2 wherein the measuring of expression levels in the first and second tests comprises measuring the expression levels of two or more biomarkers from the respective first and second groups.

15. The method of claim 2 wherein the measuring of expression levels for the first and second tests comprises measuring the expression levels of at least two biomarkers in the first group for the first test and at least one biomarker in the second group from the second test.

16. The method of claim 2, wherein the measuring of expression levels for the first test comprises measuring the under-expression of 2,3 BPGM or SBP1.

17. The method of claim 2, wherein the measuring of expression levels for the second test comprises measuring the over-expression of CPN, TP, MCM5, and NAPSA.

18. The method of claim 2, wherein the measuring of expression levels of the at least one biomarker comprises measuring the expression levels of the at least one biomarker using immunoassays.

19. The method of claim 2, wherein the sample comprises a sample selected from a group consisting of tissue, interstitial fluid, blood serum, plasma, and pulmonary fluid of the subject.

20. The method of claim 2, wherein the significant difference is an over-expression equal-to or more-than 1.5-fold when the biomarkers are one or more of CPN, TP, MCM5, and NAPSA.
